(19) United States
(12) Patent Application Publication
(10) Pub. No.: US 2006/0210129 A1
(43) Pub. Date: Sep. 21, 2006

(54) Method and apparatus for multiple labeling detection and evaluation of a plurality of particles

(75) Inventors: Michael Trendelenburg, Dossenheim (DE); Herbert Spring, Schriesheim (DE); Luiz Henrique Monteiro-Leal, Rio de Janeiro (BR); Loraine Campanati-Araujo, Rio de Janeiro (BR); Helmut Troester, Mannheim (DE)

Correspondence Address: SEED INTELLECTUAL PROPERTY LAW GROUP PLLC, 701 FIFTH AVE, SUITE 6300, SEATTLE, WA, 98104-7092, US

(73) Assignee: DEUTSCHES KREBSFORSCHUNGZENTRUM, HEIDELBERG (DE)

(21) Appl. No.: 10/522,390

(22) PCT Filed: Jul. 30, 2003
(86) PCT No.: PCT/EP03/08446

§ 371 Date: Mar. 3, 2006

Related U.S. Application Data
(60) Provisional application No. 60/399,496, filed on Jul. 30, 2002.

(51) Int. Cl. G06K 9/00; C12M 1/34

(52) U.S. Cl. 382/128; 435/287.2

(57) ABSTRACT

The invention relates to a transmission electron microscope equipped with a 2k−2k pixel area Slow Scan Cooled Charge Coupled Device Camera connected to an image processing software for generating an image of a sample. A segmentation of gold particles in the sample is achieved by the separation from specimen structure and background noise. An identification and classification of particle types is carried out according to the shape and size of the detected particles or particle pairs and finally, the gold particle distribution is visualized by the generation of false color overlay images as well as the indication of the numbers in the image.

### FIELD OF THE INVENTION

The invention relates to an apparatus and method which allows the user of a microscope to automatically detect and quantify particle distributions in biological and medical microscopic specimens.

### INTRODUCTION TO THE INVENTION

In order to visualize and identify the location of specific antibody-antigen complexes, researchers use known methods.

When working with a light microscope, it is possible to associate the antibody with fluorescent markers that, with correct illumination, reveal the position of the desired complex. Unfortunately, the maximum resolution of the light microscope is not sufficient to permit the detailed identification of the sites where the antibody-antigen complex is located.

In order to have more information, researchers need to use an electron microscope (EM) which is capable of revealing image details up to a few nanometers (10⁻⁹ m) in size. However, electron microscopes are incapable of presenting color images, as they use electrons and not photons. So, in order to visualize the antibody-antigen complex, researchers have to substitute the fluorescent marker, used in light microscopes by electron-dense particles. The most common of such electron-dense particles used are the gold particles. The fact that one needs to use electron-dense particles and observe a gray scale image in the EM can, however, become a problem, because the contrast of the gold particles is very close to that of the background structures. In other words, it becomes very difficult to identify the gold particles against a background that sometimes presents, for our eyes, almost the same gray values.

This problem is even more complicated, when one wants to observe multiple labeling. In the light microscope it is easy to identify different antibody-antigen complexes using fluorescent markers that display different colors. In the same image one can see very clearly the position of several of the fluorescent markers. This is not so easy to do in electron microscopy images. When working with gold particles, the researchers can use different particle sizes. It happens often that small particles form clusters which can be confused with a big particle by a human operator of the electron microscope. As one of the aims of the use of markers is to comparatively evaluate the number and positions of such elements in a multiple labeling experiment, the false identification or the lack of identification of particles will lead to completely wrong conclusions. A further complication is the necessity to use a higher magnification, in order to view the very small particles for example 3, 6, 10 nm in diameter, restricting the microscope field of view to very small regions of the sample.

These are the reasons why the researchers are looking for new methods and techniques that could permit a precise identification of particles over a large field of view. This possibility became real with the development of high-quality digital cameras, such as the slow-scan-cooled charge coupled device (CCD). With these new digital cameras it is possible to see in detail the microscopic images and to discriminate between very tiny contrast differences which had previously been impossible to observe. The development of digital microscopes allows the controlled movement of the field of view and the acquisition of image sequences in an oriented manner. These image sequences allow the creation of image montages that simultaneously offer an enlarged field of view and a high resolution.

Previous attempts have been made to establish automated methods of gold particle detection in electron microscopy. The prior art tools developed failed probably because they were not reliable and were incapable of multiple localization.

### OBJECTS OF THE INVENTION

It is therefore an object of the present invention to provide researchers with an apparatus and method for the fast and reliable particle detection, counting and pseudo-color overlay.

A further object of the present invention is to provide a versatile module adapted to both single and double antigen detection of 3/10 nm, 6/15 nm and 10/25 nm gold particle pairs whereby the smaller particle type of every pair can be identified as both a singular particle and as a cluster of particles. A still further object of the present invention is to provide images as a montage to allow the analysis of particles in large field images.

### DESCRIPTION OF THE FIGURES

FIG. 1 shows an overview of the electron microscope image capturing system of the invention.

FIG. 2 shows a flow diagram of sample preparation.

FIG. 3 shows a diagram of sample preparation.

FIG. 4 shows the construction of photo-montage.

FIG. 5 shows a further example of the invention.

### DETAILED DESCRIPTION OF THE INVENTION

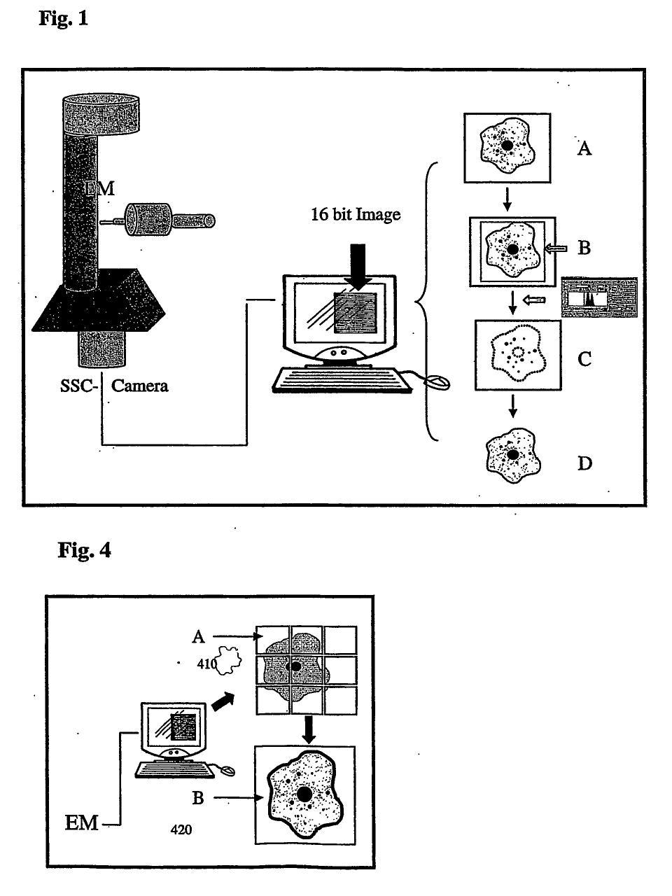

The present invention discloses a method and apparatus for double gold labeling and large field image. FIG. 1 shows an electron microscope LEO EM 912 Omega (LEO, Oberkochen, Germany) with the 2k−2k pixel array slow-scan cooled charge coupled device camera of Pro scan (SCC-CCD camera; supplied by Proscan Elektronische Systeme, Scheuring, Germany; abbreviated as “SSC-Camera” in FIG. 1). The electron microscope is used to obtain the images that are processed by the invention. The inventive module was embedded in the analysis 3.1 PRO software (SIS-Soft Imaging System, Münster, Germany) on the PC (Pentium III with 512 MB RAM). The use of the imaging equipment allows high-resolution, high-dynamic range images to be obtained that were aligned in order to produce wide-field presentations of specimen areas. After image acquisition and creation of the wide-field presentation, a user-controlled segmentation of gold particles and consequently separation from other specimen structures was performed. Following image binarization, identification and classification of the particle types is achieved through the comparison of shape and size by the inventive module according to the present invention. Afterwards, false colors were assigned to each particle group or size class.

The method described here presented several advantages. The high-dynamic range of the SSC CCD images allows the reliable separation of the gold particles from the background disregarding the contrast. Detection by means of the shape and size parameters and an evaluation for different gold particle pairs is possible using the present invention. The use of wide-field frames after multiple image alignment of high-magnification images enables the user to selectively show particle distributions in overview. Finally, the classification module allows the identification of particles that are either singular or clustered. By “clustered” is meant a very close association or agglomeration of single particles. This offers fast and reliable particle detection and evaluation of the results in research processes. An evaluation of particle distributions can be performed by the selection of a region of interest (ROI). Further, a visualization and evaluation of particle pairs in wide-field images is provided. Finally, an analysis of complete cell and tissue regions in immunocytochemical specimens at high spatial resolution becomes possible without the need for additional magnification processes.

Since gold particles or gold particle pairs of different sizes are very interesting for the purposes of microchip producing companies in their efforts to produce nanowires, it is possible that the present invention might be useful for applications in that field. In a field closer to immunocytochemistry, researchers presently working with in-situ hybridization and antigen localization mediated by antibody application or with microarrays for rapid screenings (and applying gold particles) could use the method as well. In general, the method according to the present invention helps to discriminate structures of a certain contrast and a defined size from a heterogeneous background, which, in part, exhibits comparable contrast to the target structure. The invention may be implemented as an add-on in an already existing software package. As a result the available software needs not to be changed completely but only be extended to include the modules according to the present invention.

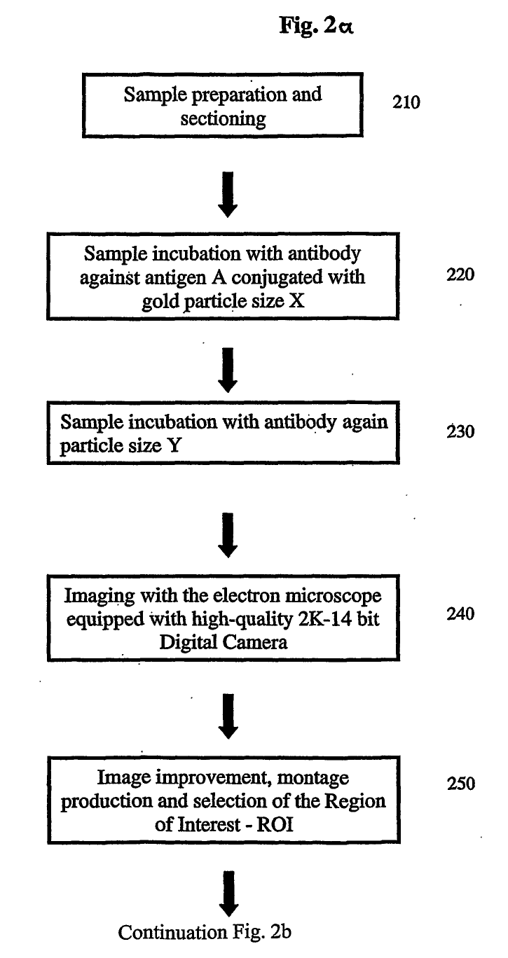

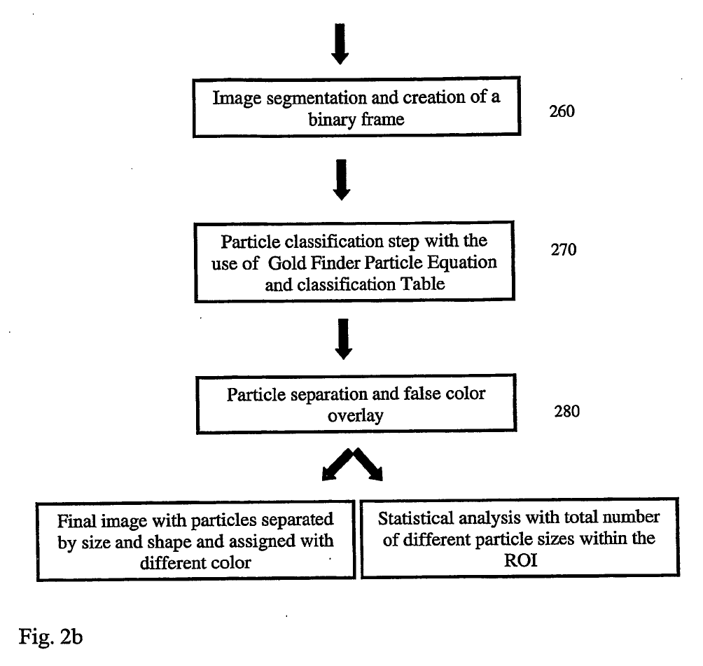

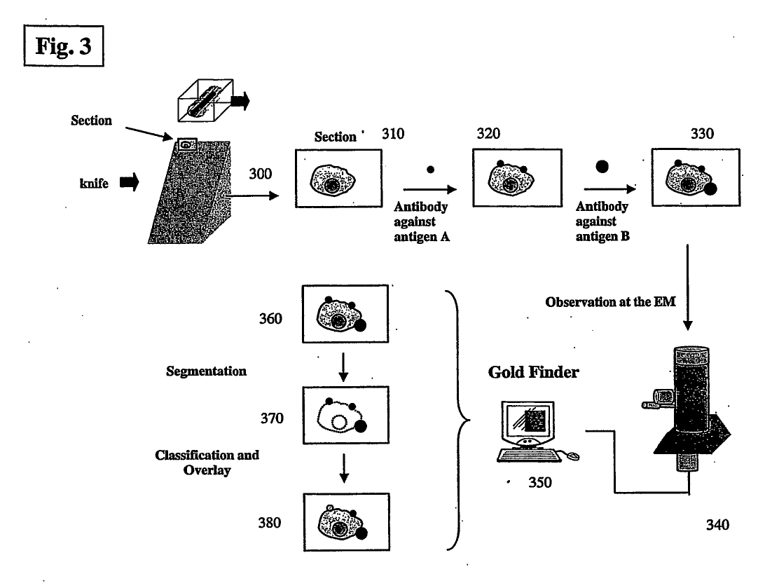

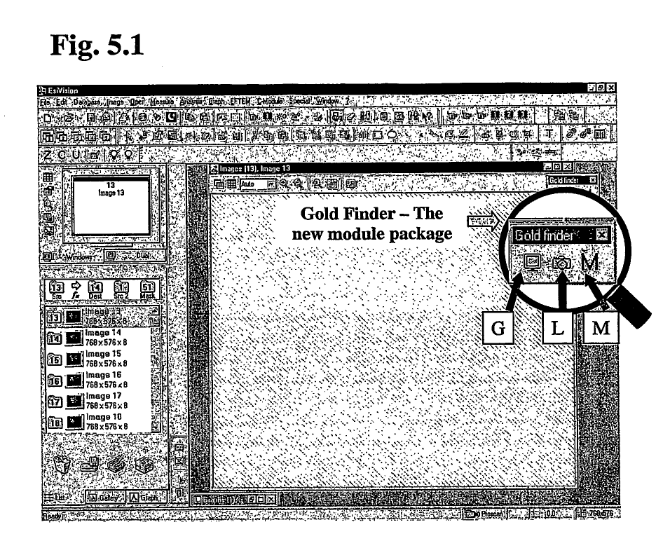

Sample preparation for observation in the microscope will now be explained with reference to FIGS. 2 and 3.

Preparation of microscopic sections.

Cells grown for 36 h were centrifuged (1500 rpm/10 min) and the pellet was directly fixed for 2 h at room temperature (or alternatively overnight at 4° C.) without washing, with a solution containing 0.1% glutaraldehyde (50% solution, grade 1; Sigma-Aldrich), 4% parafornaldehyde, 4% sucrose, 0.1% picric acid, 5 mM calcium chloride in PEM buffer (0.1 M Pipes, 2 mM EGTA, 1 mM MgSO4, pH 6.8 (Ogbadoyi et al., 2000)). After having been washed with PEM buffer, the cells were dehydrated in increasing concentrations of ethanol from 30% to 100% and embedded in the hydrophilic resin LR-White (hard grade; Agar Scientific, distributed by Plano, Wetzlar, Germany). The polymerization of LR-White blocks was performed for 48 h at 4 C under UV light irradiation. The sections (steps 300, 310) were made using a Reichert-Jung Ultracut E (Leica Mikrosysteme, Bensheim, Germany) ultramicrotome and collected on nickel grids (step 210).

The antibodies used for immunolocalization are available from commercial sources. The primary antibodies were tested by fluorescence microscopy. These were: monoclonal anti-a-tubulin from mouse, clone B 5.1.2, IgG (Sigma-Aldrich, No. T5168) used at a dilution of 1:1000; and mouse anti-actin, clone JLA 20, IgM (Oncogene, Boston, MA, USA, No. CP01), used at a dilution of 1:100. The gold-conjugated secondary antibodies used were from Aurion (distributed by Biotrend, Cologne, Germany), with different particle sizes (in this case 6 and 15 mn): goat antimouse IgG (6-nm) and goat anti-mouse IgM (15 nm), both diluted 1:40. For the 10 nm/25 nm experiments the following conjugates were used: goat anti-mouse IgG/25-nm gold and goat anti-mouse IgM/10 nm (diluted 1:40). The immunolocalization procedure was done following directions from the supplier (Aurion), with some modifications. Briefly, the sections 310 were incubated with 50 mM glycine in PBS for 15 min and nonspecific binding was prevented by incubation in 5% bovine serum albumin (BSA) plus 1% normal goat serum (NGS) for 30 min. After being washed three times in BSA-c buffer (PBS, pH 7.4, p0:1% BSA-c; Aurion, distributed by Biotrend, Cologne, Germany), the sections 310 were incubated in the primary antibody (which contained a mixture of the two antibodies) for 1 to 2 h at room temperature (step 220) as can be seen in 320. The sections 320 were then washed with BSA-c (3-10 min) and incubated with secondary antibodies (also a mixture of both gold conjugated particles, room temperature) for 1 h (step 230) as can be seen in 330. After this incubation the sections 330 were washed thoroughly with BSA-c buffer. The sections (step 330) were contrasted with an aqueous solution (5%) of uranyl acetate for 10 min, after which the grids were washed by submerging them into distilled water. The use of lead citrate was omitted. The sample was then imaged using the electron microscope 340 with the slow-scan cooled charge coupled device camera of Proscan (SCC-CCD camera (step 240)). Image acquisition and creation of the wide-field presentation was carried out in step 250. A user-controlled segmentation 360 of gold particles and consequently separation from other specimen structures was performed (step 260). Following image binarization (step 260), identification and classification 370 of the particle types are achieved through the comparison of shape and size by the present invention (step 270). Afterwards, false colors were assigned to each particle group or size class (step 280) and added as an overlay 380.

The software used to implement the invention is designed such that different windows open automatically once the previous step in the procedure was completed. The image processing will now be explained in more detail.

After the image has been acquired, the operator of the electron microscope selects a region of interest.

The image imperfections in the stored images of the selected region of interest such as uneven background are corrected by the subtraction of the original image from a background image which has been previously stored. This subtraction function uses two different reference images: a gain image and an offset image. The subtraction is carried out frame by frame. This function is to be found in the 3.1 PRO software package.

A step of improving the contrast is then carried out. This is done to ensure that the contrast and brightness of the original image is digitally increased. The stored images use a contrast histogram which is “stretched”. This function is available in the 3.1 PRO software package.

The operator can now interactively identify the range of gray scales that represents the objects of interest (i.e. the gold particle) and separates them from other background structures. The input image represents 2¹⁴ (14 bit) gray scales. The operator must teach the computer which gray scales represent the contrast of the gold particles. In order to do this, it is necessary to adjust the lower and the upper threshold values of the histogram to obtain a contrast window. With the use of a contrast window, the operator can teach the computer in what range of gray values the gold particles are included. The threshold window represents a histogram with all the gray values of an image and two vertical lines representing the upper and the lower threshold value of the grey value which limit the contrast window chosen by the operator to identify the gold particles.

It is possible on a computer screen to follow the changes in the definition of the contrast window performed by the operator. The computer creates a binary image displayed on the computer screen in which the objects of interest (gold particles) having contrast values between the threshold values will be assigned white values and the background or the other objects not of interest (i.e. not gold particles) a black value.

The original image is thus segmented into two parts: the objects of interest that appear white in the resultant image and the background and the other objects appear in black. The operator can move the contrast window and watch the target structures in the images appearing in white or vanishing, depending on the selected threshold values. When the window is set such that only the target structures (gold particles) are visible as white dots, the segmentation was complete and the subsequent steps carried out. This segmentation is necessary to ensure reliable evaluation of gold particle detection and counting. The segmentation step is provided by analysis 3.1 PRO software package (SIS-Soft Imaging System, Münster, Germany) and is shown as block 360 in FIG. 3.

The shape of the particle is now defined. This is done using the following definition of the ratio of the maximum measured diameter of the particle to the minimum measured diameter.

Sphere=Maximum diameter/minimum diameter>=0.7

Clusters=Maximum diameter/minimum diameter<0.7

In this step the computer will analyze the shape of the particles. Gold particles are usually produced with a shape substantially close to a sphere; the isolated gold particles will present a ratio of their largest to the smallest diameter of approximately 1. The particle clusters will display irregular shapes.

A step of identifying the size of the particle is then carried out. This will be explained by reference to an example working with 6 and 15 nm particle pairs.

The surface area of particles that have to be identified as gold particles by the method has to be defined. According to measurements performed prior to the invention, the limits of cross-section areas of particles for this example could be set to be

Small particles (6 nm):10 nm²<Area<60 nm²

Big particles (15 nm):40 nm²<Area<90 nm²

TABLE 1
shows a particle classification scheme developed for this example:

| Class | Area | | Correction | | |
|---|---|---|---|---|---|
| Type | Size* | Shape** | Factor (CF) | Result | Label |
| Class 1 | 10 < | Shape > | 1 | NX1 | Particle |
| | size ≦ 60 | 0.7 | | | Type 1 |
| | | | | | Small |
| Class 2 | 40 < | Shape < | 2 | NX2 | Particle |
| | size ≦ 90 | 0.7 | | | Type 1 |
| | | | | | Small |
| Class | Increment | Shape < | 3-20 | NXCF₃₋₂₀ | Particle |
| 3-20 | of 50 nm | 0.7 | | | Type 1 |
| | | | | | Small |
| Class | 90 < size | Shape > | 1 | NX1 | Particle |
| 21 | | 0.7 | | | Type 2 |
| | | | | | Big |

The final number of classes depends on the number of particle clusters.
*The Area size means cross-section area of particles and is given in nm². The microscope system must be well calibrated.
**The object shape was calculated according to the following formula: 4π (area/perimeter²);
CF₃₋₂₀ = Correction factor of classes 3-20;
N = number of objects encountered.

As a single particle or example of 6 nm diameter could have cross-sectioned area ranging from 10 to 60 nm² a range of classes was established using an increment of 50 nm² in each different class, beginning with 40 nm². If a specific object presents an area of 45 nm², it could be one single particle or a cluster of two small single particles. The method will correctly identify the object based on the shape.

These classifications are necessary because particles are produced in fact with a range of sizes near the assigned diameter, but with differences, as shown in defined Table 1. The whole range of particles is grouped according to this classification defined in the Table 1. This classification is necessary because the gold particles, especially the small ones, were not always placed as singular particles on the sample section but could be located in such a close association to one or more neighbors of gold particles that a cluster of small particles with a gold particle—like contrast but without the sphere—like shape was found. In order not to omit these and to be able to count the number of gold particles in such a cluster, the method had to be designed in such a way that these clusters could be divided up into the single particles that would be otherwise hidden in the cluster. This was again done by defining the different cross-section areas that cover a group of 2 (class 2), 3 (class 3) or more small gold particles. At the same time the shape parameter had to be defined to be different from the shape parameter of single small gold particle because the overall shape of a cluster of small particles could not be sphere-like. Therefore the definition of the shape parameter had to change from class 1 to class 2 and all the higher classes (shape>0.7 in class 1; shape<0.7; see Table 1).

In the last step the method discriminates the small particles, either as single particles or as a cluster of small particles, from the large particles. The large particles in the same specimens were observed not to cluster with each other. Therefore, in this case, it was sufficient to identify the large particles according to their cross-sectional area and having their shape close to a sphere (class 21). The number of classes, 21 in this case for the differentiation of 6 and 15 nm particles, was decided according to the observations and experience that were made during the handling of the gold particles. It was sufficient in this example for the differentiation of this pair of gold particles ( 6/15 nm) to define 21 classes of particles using the parameters as explained above.

After the identification of the particles and separation of the single particles from the clusters of small particles, and small particles from big particles, the system is able to count the total number of particles in a predetermined area. This is done by defining a correction factor for each class that was not identifying single particles. The correction factor had to be defined because the cluster of particles (i.e. classes 2 to 20) creating an area with a gold-like contrast but with irregular shape was initially identified as a single area. The correction had to be made because in the area of irregular shape a certain number of gold particles would be hidden. The number of the hidden gold particles depends on the size of the cross-section area. The total number of particles is therefore given below:
N_{T}=NS+N_{C2-20} X CF₂₋₂₀
N_{T}=Total number of particles
N_{S}=Total number of single particles
N_{C2-20}=Total number of clusters from classes 2 to 20
CF₂₋₂₀=Correction factors applied to corresponding cluster classes
CF₂₋₂₀=Correction factor for cluster evaluation

The particles separated by the method described are colorized and a final image with the original frame plus colorized particles is displayed to the operator on the computer screen as shown in block 380 on FIG. 3. A different color is used for the different particle sizes. The false colors are assigned to specific gray scale values by the image processing software. These colors are overlaid over the particles in a graphic plane and are not added permanently to the image. This result is the colorization of the particles from the gray scale image.

The number of single and cluster particle of a specific size can be summarized in the defined region of interest. In other words, depending on the region of interest selected, the system will display the total number of particles of different sizes, as well as the number of single particles and the number of cluster particles observed in the region of interest. The total number of particles in a cluster will be defined by the total area of the cluster divided by the single area of the particle, as described below.

$$Total Number of Particles = \frac{The Area of the Cluster}{The Area of a Single Particle}$$

FIG. 4 shows a further embodiment of the invention which allows the composition of a wide-field image 420 from a plurality of single, highly magnified electron microscopic images 410 adjacent to each other as a photo-montage. The option to perform image montages is a function of the software analysis package 3.1 PRO. The same particle analysis as described above resulting in a false color overlay view can be carried out. The composed image 420 still has the quality and the spatial resolution of the original images 420.

The invention was designed to count and overlay particle pairs of different sizes depending on the type of double labeling experiment performed. The invention has been described with respect to particles of sizes 6 nm and 15 mn, but could also be used with 3/10 nm, and 10/25 nm particle size pairs. The difference in size between the particles belonging to a labeling pair should be at least above 100%. This is due to a greater variability in the sizes of the colloidal gold particles. As can be seen from Table 1, the area of a single small particle of a mean diameter of 6 nm can indeed extend from 10 to 60 nm². A similar variability in size can be found for larger particles. The single particles were identified by the computer taking into account both size and shape.

Furthermore in the illustrated example, one particle with 30 nm² area and with a spherical or almost spherical shape will be classified by the method as a single small particle, whereas a particle of 120 nm² area and round shape will be identified as a big single particle. If, in this example, an object covers an area of 90 nm² and exhibits an irregular shape after the segmentation step, the computer will identify this element as a cluster of particles and assign it to the object class 2.

Should two particles be clustered together, the shape will not be spherical and the object will be identified as being composed of two small particles.

The invention also works with only one type of label in a specimen. Therefore the method is also useful for single label detection on the basis of gold particles.

The invention can also be used for any automatic label structure detection in electron microscopy. This includes wide-field detection of elemental markers that are useful for labeling on the basis of energy-filtering transmission electron microscopy (EFTEM). EFTEM allows the selective presentation of certain chemical elements in specimens. If labels are available that are designed like gold particles (spherical) and are composed of a defined chemical element, these labels can be separated and selectively displayed by the method presented here, comparable to what was described here for gold particle detection.

### EXAMPLE

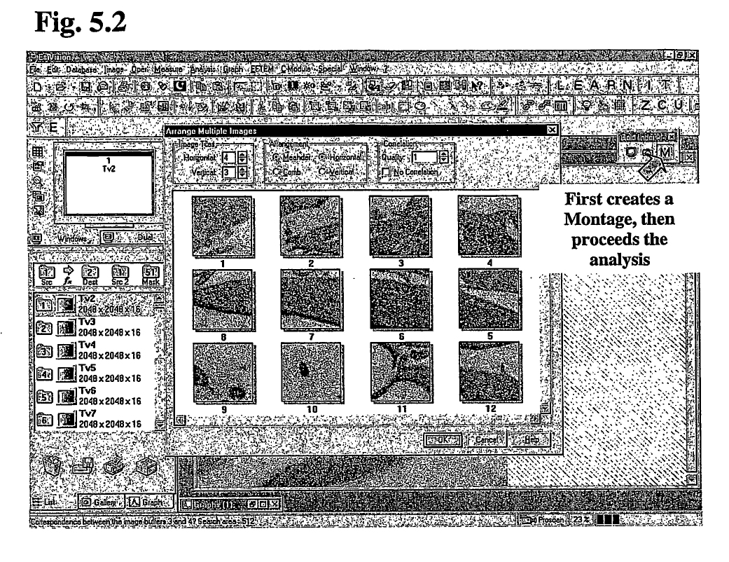

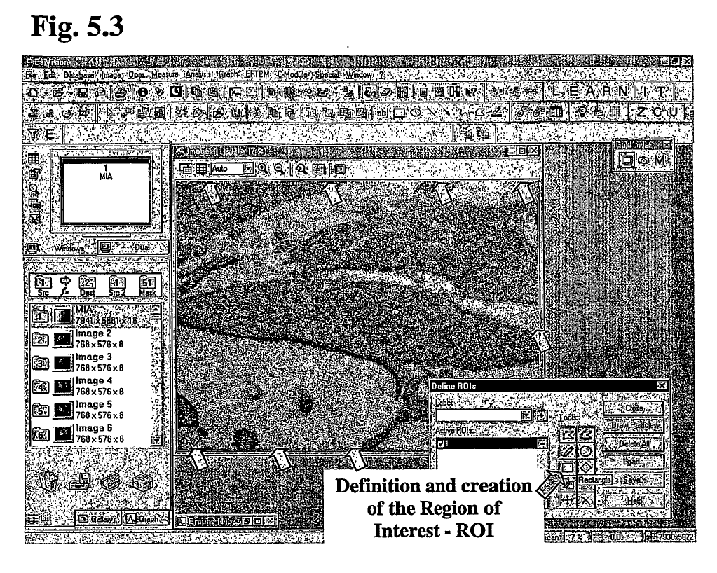

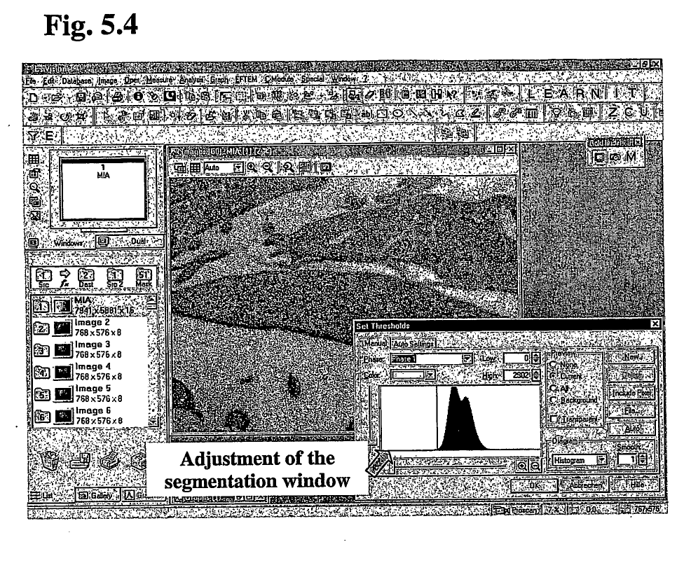

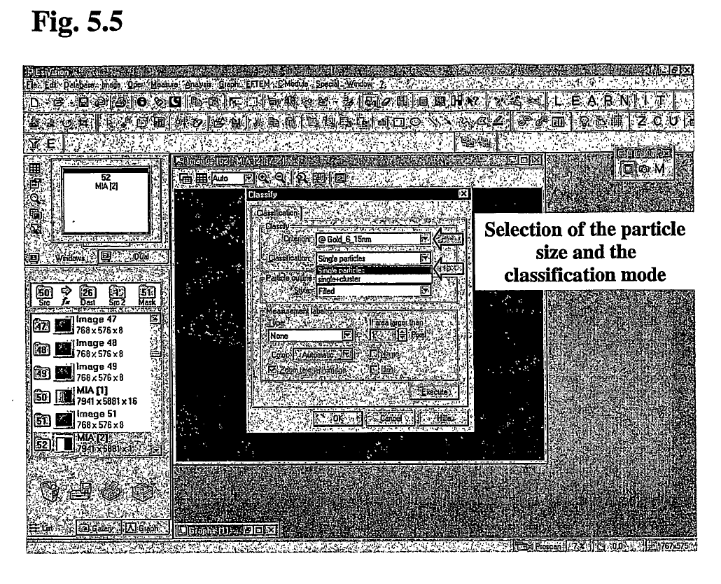

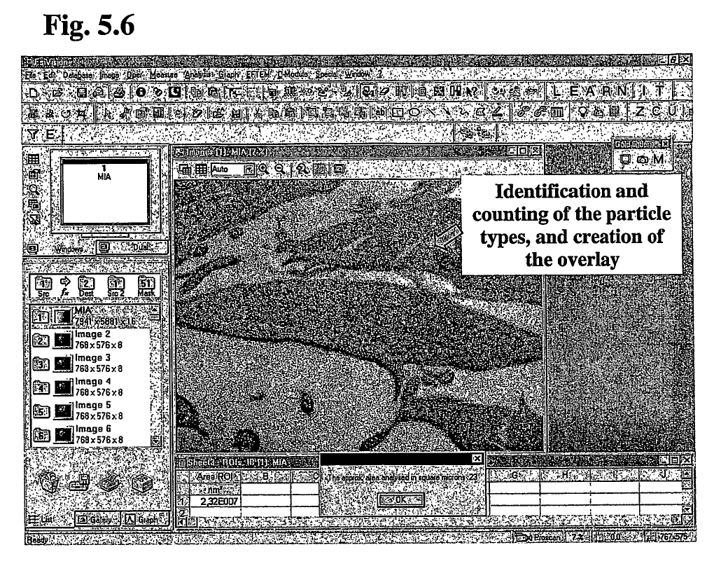

For the sake of clarity the invention was described herein using a single sample image. The following FIG. 5.1 to 5.6 display an actual example of the invention in which the image montage on the computer screen is constructed.

FIG. 5.1 shows the invention having three options. Processing the image from an image store or gallery (labeled G), acquiring the image from the microscope (L) or first creating a montage (M) of several images and then proceeding with the analysis.

FIG. 5.2 shows the first step within the montage option, the acquisition and alignment of several high resolution (2048 square pixel size) and high dynamic range (14 bit) images.

In FIG. 5.3, after the automatic conclusion of the montage, the method allows the operator to define one region of interest (ROI—defined by a frame). In this example the whole image is selected.

In FIG. 5.4 the contrast window is established. The thin line in the contrast window should only reach the beginning of the histogram in the image, i.e. the initial planar region (left part in the contrast window, at the crossing point to the left black peak).

FIG. 5.5 shows the selection of the particle size and the classification mode. In this step the operator decides the size of the particle pair used ( 3/10 nm, 6/15 nm or 6/25 nm) and a if the detection will be limited to single particles or to single cluster particle detection. In the latter case, the computer will estimate the number of particles belonging to a cluster depending on the total cluster area.

In FIG. 5.6, the number of particles is counted and an overlay image created.

1. Method for multiple labeling detection and evaluation of a plurality of particles in specimen analysis comprising the steps of:
i) Acquisition of at least one image in a single color scale;
ii) A separation of the plurality of particles using a color threshold;
iii) Identification and classification of one or more particle types of the plurality of the particles based on the size and shape of the plurality of the particles to form different particle classes; and
iv) Visualization of locations of the plurality of particles.

2. Method of claim 1, wherein the acquisition of contrast images is made by a slow scan cooled charge couple device camera.

3. Method of claim 2, wherein the images are at least 14 bit images.

4. Method of claim 1, wherein the separation of the plurality of particles is carried out with reference to an underlying background image.

5. Method of claim 1, wherein the separation of the plurality of particles is carried out with reference to specimen structures.

6-7. (canceled)

8. Method of claim 1, wherein the images are processed using at least 16 bits.

9. Method of claim 1, wherein the image can be a photo-montage of a plurality of the images.

10. Method of claim 1, wherein the step of visualization of the location of the plurality of particle is carried out by a false coloring of the plurality of particles.

11. Method of claim 10, wherein different false colors are used for different particle classes.

12. Method of claim 1, further comprising the generation of an overlay image.

13. Apparatus for the analysis of particles comprsing:
an image capture device for capturing a single color image of a speciment with a plurality of particles;
an image enhancement device;
an image identification and classification device for classifying the plurality of particles based on the size and shape of the plurality of the particles; and
a display device for visualizing the specimen with the particles.

14. Apparatus of claim 13, wherein the image capture device is a camera connected to an electron microscope.

15. Apparatus of claim 14, wherein the camera is a slow scan charge coupled device.

16-17. (canceled)

18. Apparatus of claim 13, wherein the display devices visualize the particles in false colors.
